(19) United States
(12) Patent Application Publication
(10) Pub. No.: US 2004/0039357 A1
(43) Pub. Date: Feb. 26, 2004

(54) Arrangement at a stoma cuff

(76) Inventor: Svein Jakob Andersen, Randaberg (NO)

Correspondence Address: KNOBBE MARTENS OLSON & BEAR LLP, 2040 MAIN STREET, FOURTEENTH FLOOR, IRVINE, CA, 92614, US

(21) Appl. No.: 10/362,199

(22) PCT Filed: Aug. 20, 2001
(86) PCT No.: PCT/NO01/00341

(30) Foreign Application Priority Data
Aug. 21, 2000 (NO) 20004174

(51) Int. Cl. A61F 5/44

(52) U.S. Cl. 604/332

(57) ABSTRACT

An arrangement at a stoma plate (15) for use with persons or animals with a colostoma (10), where there is a collecting device in the form of a stoma plate (15) and a collecting bag (19) or a similar arrangement, and where a stoma cuff (1) is disposed to enclose and seal against the colon (10), while at the same time being connected to the stoma plate (15) or another part, possibly of a similar collecting device, in a sealing manner.

This invention regards a cuff for use by persons with a colostoma. The cuff is designed to prevent stool from getting into contact with the surrounding skin in the area where the colon discharges.

A considerable number of people have undergone operations for injuries and diseases that involve the colon having to be led out through the abdominal wall. This solution entails stool issuing from the colon being collected in a bag.

Several almost identical solutions for collecting stool are available. All the solutions entail a plate, a so-called stoma plate normally made from non-rigid plastic and equipped with a central opening adapted to the protruding end of the colon, being guided over the colon and glued to the abdominal skin by the colon by means of an adhesive. On the side facing away from the skin, the plate is provided with an annular bead that seals against the plate. The bead profile varies with the various suppliers' products, but in all cases it is designed to form a fastening for a collection bag for stool, a so-called stoma bag.

In such a more or less standardised solution, the outside of the projecting colon is exposed to stool that to some extent is rather corrosive. A further problem is that stool gets in along the colon to the skin underneath the stoma plate. Such contact between skin and stool will over time cause sore skin and pains. It is known that special creams applied in the area between the butt-end of the projecting colon and the central hole may counteract leaks. However the solution is not satisfactory, and users find that they can not lead a normal life because the soreness of the skin around the colostoma prevents fastening of an ordinary stoma plate.

The object of the invention is to remedy the negative aspects of prior art.

The objective is achieved in accordance with the invention by the characteristics stated in the description below and in the following claims.

A stoma cuff in the form of the frustum of a cone in a relatively soft material is guided inwards over the protruding end of the colon, its tapering end portion sealing against the outer periphery of the colon. The bottom part of the cone is provided with a projecting annular flange-like plate, which on the side facing away from the tapering part of the cone is coated with adhesive. The adhesive side of the flange-like plate is pressed against and glued in a sealing manner to the outside of the stoma plate, thus forming a seal between the colon and the stoma plate, preventing this stool from getting past the opening for leading the colon through the stoma plate, and further to the skin behind it.

Several embodiments of the stoma cuff and methods of fastening may be used, and the most relevant are discussed below, with reference to figures.

The following describes several non-limiting examples of preferred embodiments illustrated in the accompanying drawings, in which:

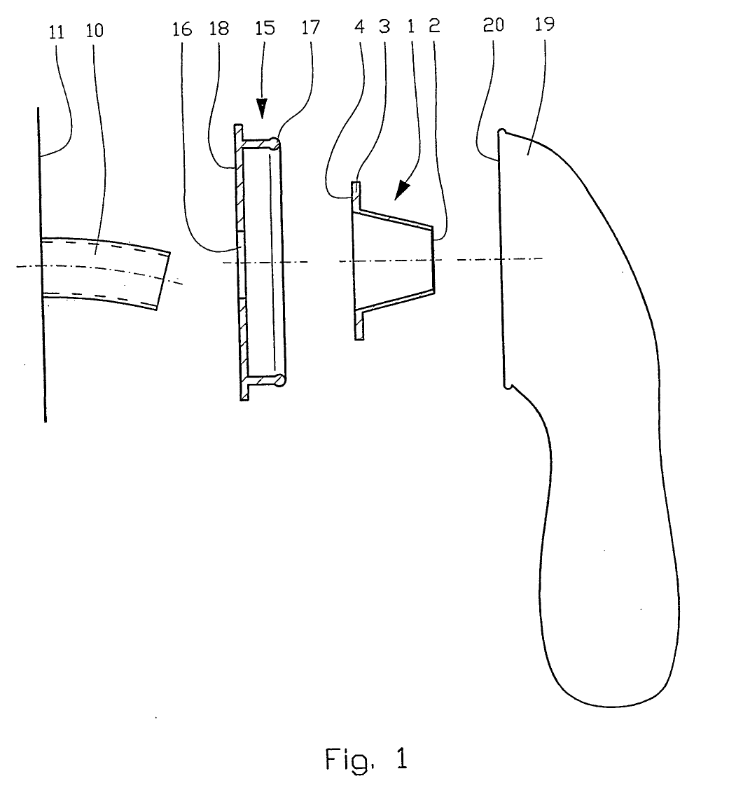

FIG. 1 is a sectional and exploded view of the parts that enter into a collection system;

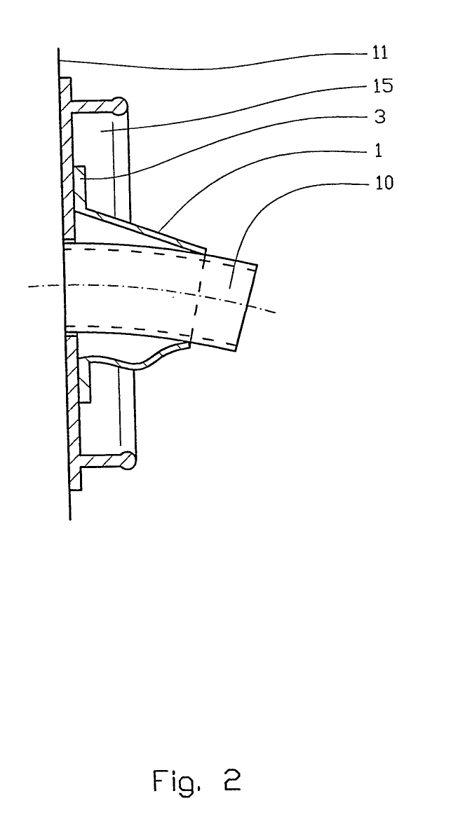

FIG. 2 is a sectional view of the components of FIG. 1 in the operational position;

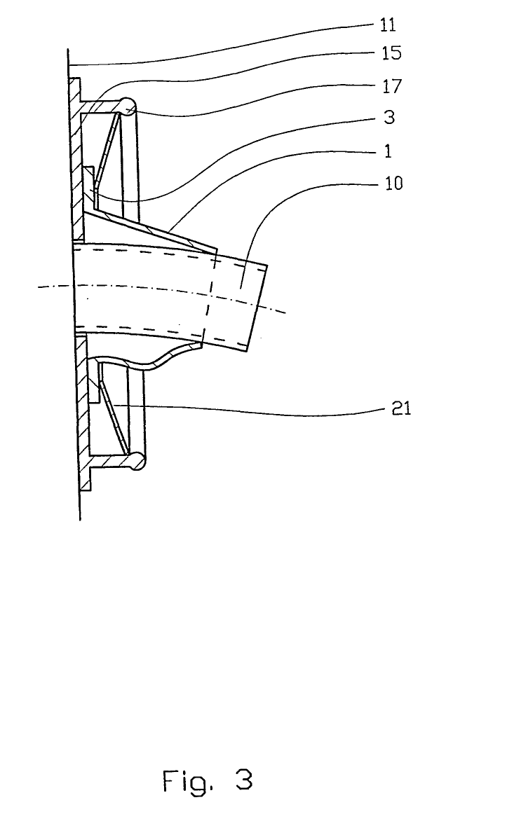

FIG. 3 is a sectional view of an alternative method of fastening by use of a clamping ring;

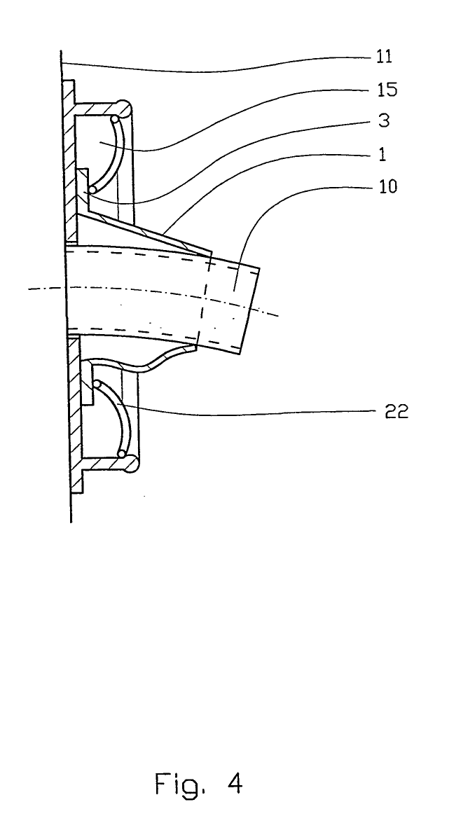

FIG. 4 is a sectional view of an alternative method of fastening by use of a clip;

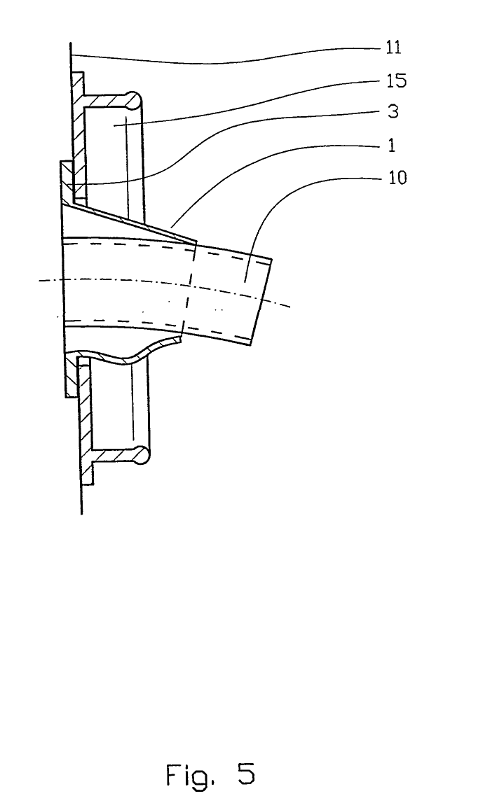

FIG. 5 is a sectional view of an alternative method of fastening by use of the actual stoma plate as a fastening element;

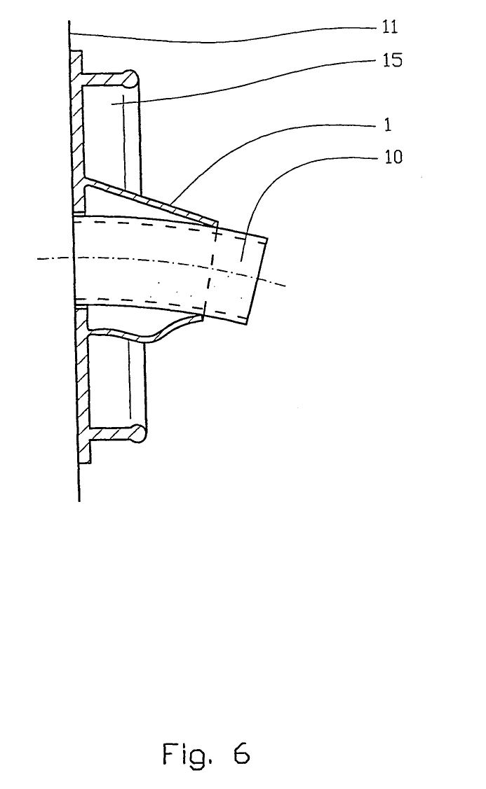

FIG. 6 is a sectional view of an alternative method of fastening in which the stoma cuff forms an integrated part of the stoma plate;

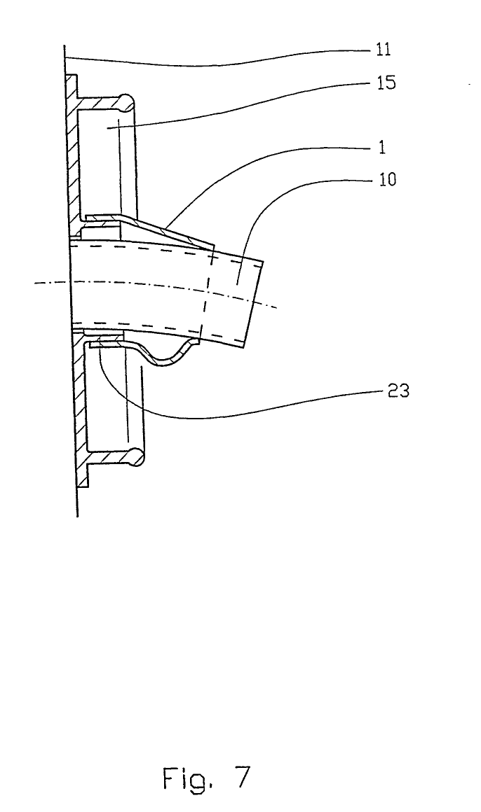

FIG. 7 is a sectional view of an alternative method of fastening in which the stoma cuff is guided onto a mounting part protruding from the stoma plate; and

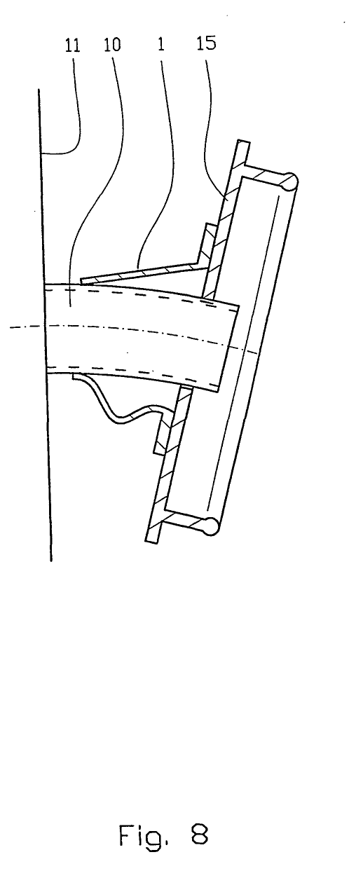

FIG. 8 is a sectional view of the stoma cuff attached in the reverse direction.

In the drawings, reference number 1 denotes a stoma cuff according to the invention, see FIG. 1. The tapering end portion of the stoma cuff 1, which is preferably manufactured in a relatively soft material, is provided with a through opening 2, and the bottom end portion is provided with an encircling circular plate forming a flange facing 3. The flange facing 3 may be an integral part cast with the stoma cuff 1 or a separate unit attached to the stoma cuff 1. The side of the flange facing 3 which faces away from the tapering end portion of the stoma cuff 1 is coated with an adhesive 4.

On a stoma-operated person, a colon 10 normally protrudes from the abdominal skin 11. A round or angular stoma plate 15 is coated with an adhesive 18 on the side that faces the skin 11 of the user, and has an essentially central opening 16, relative to the plate 15. To the outside of the stoma plate 15 is sealingly attached an annular bead 17 that fits and seals against an attachment bead 20 in the opening section of a stoma bag 19.

In use, the opening 16 of the stoma plate 15 is, according to prior art, slipped over the colon 10. The opening 16 is pre-adjusted to fit the cross-sectional shape and size of the colon 10, see FIG. 2. The adhesive 18 of the stoma plate 15 adheres to the skin 11. According to the invention, the stoma cuff 1 is slipped over the colon 10, whereby the tapering end portion 2 of the stoma cuff encloses the colon in a sealing manner. The adhesive 4 of the stoma cuff 1 is pressed against the outside of the stoma plate 15, adhering to the stoma plate 15 in a sealing manner. Thus, a leakproof seal is formed between the colon 10 and the stoma plate 15.

The attachment bead 20 of the stoma bag 19 is guided in over the annular bead 17 of the stoma cuff 15 in a known manner, forming a leakproof joint between the stoma-plate 15 and the stoma bag 19.

In another embodiment, see FIG. 3, the stoma cuff 1 is fastened to the stoma plate 15 by means of a clamping ring 21. The clamping ring may be given several appropriate shapes, however in the figure it is shown as a disc-like tension element bracing between the bead 17 and the flange facing 3.

In a further embodiment, the stoma cuff 1 is fastened to the stoma plate 15 by a clip 22 bracing between an attachment point in the stoma plate 15 and the flange facing 3. The attachment point for clips of various types in the stoma plate 15 may be given other appropriate shapes, such as bevels, lips, apertures or enclosing grooves. The stoma cuff 1 may also be guided in from the adhesive side of the stoma plate 15, see FIG. 5.

In yet another embodiment, the stoma cuff 1 may form an integral part of the stoma plate 15 or other type of collecting device used for the same purpose, and be manufactured together with the collecting device, see FIG. 6.

In a further embodiment, the stoma cuff 1 is, in an enclosing manner, slipped over a collar 23 projecting from the stoma plate, see FIG. 7.

The shape of the stoma cuff 1 is not limited to the conical shape shown in FIGS. 1 to 7, but may assume other appropriate shapes such as disc, bellows, sleeve or curved designs. The function of the stoma cuff is served by it being placed against the colon 10 in an enclosing and sealing manner, whilst sealing against the stoma plate 15 or other collecting device.

When the user's skin is sore and the mounting of a stoma plate 15 to the skin 11 is undesirable, it is possible to turn the cuff 1 round, so as to make it project from the rear of the stoma plate 15 and enclose the colon 10, see FIG. 8.

1. An arrangement at a stoma plate (15) for use with persons or animals with a colostoma (10), where there is a collecting device in the form of a stoma plate (15) and a collecting bag (19) or a similar arrangement, characterised in that a stoma cuff (1) is disposed so as to seal against the colon (10) around a circumferential line of contact where the circumferential line of contact is arranged to move with the colon (10) as the colon (10) expands and contracts, while at the same time being connected to the stoma plate (15) or another part, possibly of a similar collecting device, in a sealing manner.

2. An arrangement in accordance with claim 1, characterised in that the stoma cuff (1) has the form of a truncated cone.

3. An arrangement in accordance with one or more of the preceding claims, characterised in that one end portion of the stoma cuff (1) is provided with a flange-like facing (3).

4. An arrangement in accordance with one or more of the preceding claims, characterised in that the fastening side of the flange-like facing (3) is coated with an adhesive (4).

5. An arrangement in accordance with one or more of the preceding claims, characterised in that the stoma cuff (1) is welded to the stoma plate (5).

6. An arrangement in accordance with one or more of the preceding claims, characterised in that the stoma cuff (1) is fastened to the stoma plate (5) by a clamping ring (21).

7. An arrangement in accordance with one or more of the preceding claims, characterised in that the stoma cuff (1) is fastened to the stoma plate (5) by one or more clips (22).

8. An arrangement in accordance with one or more of the preceding claims, characterised in that the stoma cuff (1) is fastened to the stoma plate (5) at a projecting collar (23).
